(19) United States
(12) Patent Application Publication
(10) Pub. No.: US 2025/0230222 A1
(43) Pub. Date: Jul. 17, 2025

(54) ANTIBODIES FOR TREATMENT OF CORONAVIRUS DISEASE

(71) Applicants: Northwestern University, Evanston, IL (US); The Texas A&M University System, College Station, TX (US)

(72) Inventors: Huiping Liu, Evanston, IL (US); Deyu Fang, Evanston, IL (US); Hannah Faisal Mubarak, Evanston, IL (US); Andrew Daniel Hoffmann, Evanston, IL (US); Nurmaa Khund Dashzeveg, Evanston, IL (US); Lamiaa Khamies Mohamed Ali El-Shennawy, Evanston, IL (US); Juncheng Wei, Evanston, IL (US); Yang Shen, College Station, TX (US); Wuwei Tan, College Station, TX (US)

(21) Appl. No.: 19/000,508

(22) Filed: Dec. 23, 2024

Related U.S. Application Data
(60) Provisional application No. 63/613,636, filed on Dec. 21, 2023.

(51) Int. Cl. C07K 16/10; A61K 39/00; A61P 31/14

(52) U.S. Cl. CPC C07K 16/1003; A61P 31/14; A61K 2039/505; C07K 2317/76; C07K 2317/92

(57) ABSTRACT

Provided herein are antibodies and pharmaceutical compositions for treating coronavirus disease, and methods of their use.

### CROSS REFERENCE TO RELATED APPLICATIONS

This application claims the benefit of and priority to U.S. Application Ser. No. 63/613,636, filed Dec. 21, 2023. The entirety of which is incorporated herein by reference.

### STATEMENT REGARDING FEDERALLY SPONSORED RESEARCH OR DEVELOPMENT

This invention was made with government support under grant numbers GM124952, AI167272, and AI167272 awarded by the National Institutes of Health. The government has certain rights in the invention.

### REFERENCE TO AN SEQUENCE LISTING

The content of the electronic sequence listing (File name: 702581_02587.xml; Size 16,874 bytes; and Date of Creation: Dec. 23, 2024) is herein incorporated by reference in its entirety.

### BACKGROUND

During the pandemic of coronavirus disease 2019 (COVID-19), neutralizing antibodies became one of the earliest approaches that rapidly developed and effectively treated the early phase of infections before a vaccine was developed and before the coronavirus evolved to evade neutralization (8-12). Most of the coronavirus neutralizing antibodies disrupt the spike interactions with human ACE2 thereby preventing viral entry for prophylactic and therapeutic applications (8-15). The constantly-evolving viral sequences especially the variants of concern (Delta and Omicron) started to escape from neutralizing antibodies and vaccination immunity (16, 17), partially contributing to the death of nearly seven million people worldwide (18). The continuous development of neutralizing antibodies against the ever-evolving variants is of interest.

### SUMMARY

In a first aspect, disclosed herein is an antibody comprising: a variable heavy chain (VH) comprising SEQ ID NO: 11 or a sequence having at least 90% identity thereto; and a variable light chain (VL) comprising SEQ ID NO: 12 or a sequence having at least 90% identity thereto; wherein the antibody comprises at least one mutation at a position selected from: position 77 relative to SEQ ID NO: 11; position 53 relative to SEQ ID NO: 12; position 70 relative to SEQ ID NO: 12; and position 71 relative to SEQ ID NO: 12. The VH may comprise a lysine, an asparagine, or a cysteine at position 77 relative to SEQ ID NO: 11. The VL may comprise a serine, an alanine, a glutamine, or a glycine at position 53, relative to SEQ ID NO: 12. The VL may comprise an asparagine, or an alanine at position 70 relative to SEQ ID NO: 12. The VL may comprise a cysteine at position 71 relative to SEQ ID NO: 12. The VH may comprise SEQ ID NO: 1, 2, or 3 or a sequence having at least 90% identity thereto. The VL may comprise SEQ ID NO: 4, 5, 6, 7, 8, 9, or 10 or a sequence having at least 90% identity thereto.

In another aspect, provided herein is a pharmaceutical composition comprising a therapeutically effective amount of at least one antibody described herein; and a pharmaceutically acceptable carrier.

In another aspect, provided herein is a method for treating coronavirus disease in a subject in need thereof, the method comprising administering to the subject a pharmaceutical composition described herein. The coronavirus disease may be caused by a SARS-CoV-2 Delta variant.

### BRIEF DESCRIPTION OF THE DRAWINGS

The patent or patent application file contains at least one drawing in color. Copies of this patent or patent application publication with color drawings will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A shows a schematic describing study flow and AbGen platform. Peripheral blood samples were collected from 42 convalescent Covid-19 patients and RBD+ memory B-cells were sorted and underwent single-cell VDJ sequencing using the 10× Genomic platform.

FIG. 1B shows a schematic describing experimental analyses of 19/1366 and 10 re-designed IgGs for virus neutralization. Prioritized antibodies were then tested for their ability to neutralize broad-spectrum SARS-CoV-2 in both in-vitro and in-vivo settings.

FIG. 2A shows frequencies of V genes sequenced from 1366 RBD-bound B cells, within the antibody heavy (H) and Light chains (Kappa (K), and Lambda (L).

FIG. 2B shows frequencies of J genes sequenced from 1366 RBD-bound B cells, within the antibody heavy (H) and Light chains (Kappa (K), and Lambda (L).

FIG. 2C shows frequencies of D genes sequenced from 1366 RBD-bound B cells, within the antibody heavy (H) and Light chains (Kappa (K), and Lambda (L).

FIG. 2D shows frequency of C genes in H, K, and L chain sequences.

FIGS. 2E-2F show frequency of CDR3 sequence in H, K, and L chains of VDJ sequences retrieved from the RBD-bound B cells of patients recovered from early wild-type SARS-CoV-2 infection.

FIG. 3A shows a schematic depicting experimental workflow. Variable regions of the heavy and light chains of the antibodies were cloned into AbVec IgG vectors followed by transfection into 293T cells. Purified antibodies were then tested for their ability to neutralize RBD in an ACE-2 cell-binding assay as well as live SARS-CoV2 neutralization.

FIG. 3B-3C shows live SARS-CoV-2 virus neutralization by anti-RBD antibodies. Viability of A549 cells overexpressing human ACE2 was evaluated 72-96 hours post viral infection. GraphPad Prism 9.3.1 was used to calculate EC50s.

FIG. 4A shows antibody neutralization of recombinant RBD in an ACE2+ HEK-293 cell-based neutralization assay. Increasing anti-RBD antibody concentrations were incubated with recombinant wildtype RBD followed by the addition of ACE+ overexpressing HEK-293T cells. The amount of non-neutralized RBD available to bind to ACE2 overexpressing cells was then followed using a flowcytometry approach.

FIG. 4B shows live SARS-CoV-2 virus neutralization by anti-RBD antibodies. Antibody dilutions were pre-incubated with 500 pfu of SARS-CoV-2 Omicron B.5 variants. Following incubation, antibody-virus mix was added to A549 cells overexpressing human ACE2. At 96 hours post infection, cells were fixed, and cell viability was determined.

FIG. 5A shows antibody 106/107 redesign with improved efficacy against delta variant. FIG. 5A shows visualization of the binding interface between the spike RBD (in wheat cartoon), including two positions mutated in the Delta variant (L452 and T478), and the patient antibody 106/107 (in blue cartoons). Four positions on the antibody were selected for redesign and shown in sticks, including F66 of the heavy chain (in darker blue) and Y38, Y55, and D56 of the light chain (in lighter blue).

FIG. 5B shows live SARS-CoV-2 virus neutralization by our redesigned antibodies. Data showing fold-improvement of EC50 for the redesigned antibodies compared to the WT 106/107 antibody. Viability of A549 cells overexpressing human ACE2 was evaluated 72-96 hours post viral infection. GraphPad Prism 9.3.1 was used to calculate EC50s.

FIG. 5C shows computational designed single amino-acid substitutions of antibody 106/107: computationally-predicted binding-energy improvements (−ddG) showed high correlations with experimentally-measured EC50 fold improvements, whether tested against WT (Washington) or the Delta variant.

FIG. 5D shows computationally predicted 3D structures of RBD-Ig complexes suggested the WT-neutralizing mechanism of the antibody 106/107 (where Y38 (L) and D56 (L) interacted with T478 on the RBD wild type), the antibody-escaping mechanism of the Delta variant (where Y38 (L) and D56 (L) lost interactions with K478 on the RBD Delta variant and Y38 (L) paid more desolvation penalty), and the variant-neutralization enhancing mechanism (where A38 (L) paid almost no desolvation penalty).

FIG. 6A shows live SARS-CoV-2 virus neutralization for Washington variant mutated anti-RBD antibodies at increasing antibody concentrations. Percent neutralization of virus infecting A549 cells.

FIG. 6B shows live SARS-CoV-2 virus neutralization for Delta variant mutated anti-RBD antibodies at increasing antibody concentrations. Percent neutralization of virus infecting A549 cells.

FIG. 7A shows antibody neutralization and inhibition of broad strain infections in hACE2 transgenic mice. Schematic of viral neutralization and infection design. SARS-CoV-2 viral strains and select antibodies (1.2 ug IgG for Washington and Delta or 8.5 ug IgG for Omicron) were administered intranasally into K18-hACE2 mice. Animals were monitored for health twice daily and weighed once per day. Seven days post infection (3 days for Omicron), mice were euthanized, and lungs were analyzed for viral load and pathohistological characteristics.

FIG. 7B shows percent weight loss of K18-hACE2 mice post viral infection (n=9).

FIG. 7C shows clinical score post SARS-CoV-2 infection. Larger clinical scores indicate increased disease severity and reduced physical fitness (n=9).

FIG. 7D shows lung genomic viral load on day 7 (Washington and Delta) or day 3 (Omicron) post antibody neutralization and viral infection measured by qRT-PCR (n=9).

FIG. 7E shows representative H&E images of mice lungs at experimental endpoint. Error bar=1 mm.

FIG. 7F shows lung acute and chronic inflammation scores based on histopathological analysis of H&E-stained slides (n=9).

FIG. 7G shows alveolar hemorrhage and necrosis scores based on histopathological analysis of H&E-stained slides (n=9). Statistical significance was tested with unpaired t-tests (b, c, f and g) or Mann Whitney test (d). ns means non-significant difference.

FIG. 8A shows lung histology inflammation and hemorrhage scores for Washington (WT). Alveolar hemorrhage and necrosis scores based on histopathological analysis of H&E-stained slides (n=9). Statistical significance was tested using unpaired t-tests. ns means non-significant difference.

FIG. 8B shows lung histology inflammation and hemorrhage scores for Delta variant. Alveolar hemorrhage and necrosis scores based on histopathological analysis of H&E-stained slides (n=9). Statistical significance was tested using unpaired t-tests. ns means non-significant difference.

FIG. 8C shows lung histology inflammation and hemorrhage scores for Omicron variant. Alveolar hemorrhage and necrosis scores based on histopathological analysis of H&E-stained slides (n=9). Statistical significance was tested using unpaired t-tests. ns means non-significant difference.

FIG. 9 shows lung histology images. Representative H&E images of mice lungs at the experimental endpoint.

FIG. 10 shows a sequence alignment between antibody 106/107 (H/L chains with CDR3 highlighted in yellow) and a clinical antibody CD144.

### DETAILED DESCRIPTION

Coronavirus disease 2019 (COVID-19) is a contagious disease caused by the coronavirus SARS-CoV-2. There are many thousands of SARS-CoV-2 variants grouped together into clades or lineages. The Delta variant (B.1.617.2) is one of the dominant variants spreading among global populations. Therapeutic antibodies have become some of the most influential therapeutics in modern medicine to fight against infections pathogens, cancer, and many other diseases. Through computational methods, the inventors have identified novel antibodies having improved efficacy against the SARS-CoV-2 Delta variant.

In a first aspect, provided herein is an antibody comprising: a variable heavy chain (VH) comprising SEQ ID NO: 11 or a sequence having at least 90% identity thereto; and a variable light chain (VL) comprising SEQ ID NO: 12 or a sequence having at least 90% identity thereto; wherein the antibody comprises at least one mutation at a position selected from: position 77 relative to SEQ ID NO: 11; position 53 relative to SEQ ID NO: 12; position 70 relative to SEQ ID NO: 12; and position 71 relative to SEQ ID NO: 12.

In embodiments, the VH comprises a lysine, an asparagine, or a cysteine at position 77 relative to SEQ ID NO: 11. In embodiments, the VL comprises a serine, an alanine, a glutamine, or a glycine at position 53, relative to SEQ ID NO: 12. In embodiments, the VL comprises an asparagine, or an alanine at position 70 relative to SEQ ID NO: 12. In embodiments, the VL comprises a cysteine at position 71 relative to SEQ ID NO: 12.

The VH may comprise SEQ ID NO: 1, 2, or 3 or a sequence having at least 90% identity thereto; and the VL comprises SEQ ID NO: 4, 5, 6, 7, 8, 9, or 10 or a sequence having at least 90% identity thereto, wherein the antibody comprises at least one of: a lysine, an asparagine, or a cysteine at position 77 of the VH, a serine, an alanine, a glutamine, or a glycine at position 53 of the VL, an asparagine, or an alanine at position 70 of the VL, and a cysteine at position 71 of the VL.

The terms “antibody” and “antibody molecule” are used herein interchangeably and refer to immunoglobulin molecules or other molecules which comprise an antigen binding domain. Antibodies include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g. ability to bind to the protein of interest or variant) are retained.

The term antibody includes “antibody fragments” or “antibody-derived fragments” and “antigen binding fragments” which comprise an antigen binding domain and displays antigen binding function, for example, Fab, Fab′, F(ab′)2, scFv, Fv, dsFv, ds-scFv, Fd, mini bodies, monobodies, and multimers thereof and bispecific antibody fragments. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

As mentioned above, fragments may comprise a heavy chain variable region (Vu domain) and light chain variable region (V_{L}). Fragments may comprise one or more of the heavy chain complementarity determining regions (CDRHs) of the antibodies or of the V_{H} domains, and one or more of the light chain complementarity determining regions (CDRLs), or V_{L} domains to form the antigen binding site.

The terms “complementarity determining region” and “CDR” refer to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site. Since the antigen binding sites are typically composed of two variable domains (on two different polypeptide chains, heavy and light chain), there are six CDRs for each antigen binding site that can collectively come into contact with the antigen. A single whole antibody molecule has two antigen binding sites and therefore contains twelve CDRs. For further example, sixty CDRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies.

The human V_{H} complex is composed of approximately 100 gene segments per haploid genome, including at least 51 functional genes, as judged by successful rearrangement in cloned cDNA. On the basis of nucleic acid sequence homology, the V_{H} genes have been grouped into 6-7 families (VH 1-7). Among the seven families, the VH3 family is the largest.

Antibodies can be genetically engineered from the CDRs, VH, VL, and monoclonal antibody sequences described herein into antibodies and antibody fragments by using conventional techniques such as, for example, synthesis by recombinant techniques or chemical synthesis. Techniques for producing antibody fragments are well known and described in the art.

One may wish to engraft one or more CDRs from the monoclonal antibodies described herein into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to a new scaffold all of the previous modifications described can also be performed. For example, one could consult Biotechnol Genet Eng Rev, 2013, 29:175-86 for a review of useful methods.

The antibodies or antibody fragments can be wholly or partially synthetically produced. Thus, the antibody may be from any appropriate source, for example recombinant sources and/or produced in transgenic animals or transgenic plants. Thus, the antibody molecules can be produced in vitro or in vivo. The antibody or antibody fragment can be made that comprises all or a portion of a heavy chain constant region, such as an IgG1, IgG2, IgG3, IgG4, IgA1, IgA2, IgE, IgM or IgD constant region.

Furthermore, the antibody or antibody fragment can further comprise all or a portion of a kappa light chain constant region or a lambda light chain constant region. All or part of such constant regions may be produced wholly or partially synthetic. Appropriate sequences for such constant regions are well known and documented in the art.

The terms “monoclonal antibody” and “monoclonal antibody composition” refer to a preparation of antibody molecules of a single amino acid composition that specifically binds to a single epitope of an antigen.

The term “chimeric antibody” refers to an antibody comprising a variable region, i.e., binding region, from one source or species and at least a portion of a constant region derived from a different source or species, usually prepared by recombinant DNA techniques. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. The antibodies described herein may be chimeric antibodies including heavy chain constant domains from non-human mammals (e.g., mouse, rat, rabbit, or non-human primate). The antibodies described herein may be chimeric antibodies including constant regions from rabbit heavy chain immunoglobulin sequences. Suitable heavy chain constant region sequences from non-human mammals, including mouse, rat, rabbit, and non-human primate are known in the art.

Antibodies can be made by well-known methods, such as described in Harlow and Lane, Antibodies; A Laboratory Manual, Cold Spring Harbor Laboratory, Cold Spring Harbor, N.Y., (1988). Monoclonal antibodies can be produced by immunizing inbred mice with a peptide antigen. The mice may be immunized by the IP or SC route in an amount and at intervals sufficient to elicit an immune response. The mice may receive an initial immunization on day 0 and be rested for about 3 to about 30 weeks. Immunized mice may be given one or more booster immunizations of by the intravenous (IV) or subcutaneous (SC) route. Lymphocytes, from antibody positive mice may be obtained by removing spleens from immunized mice by standard procedures known in the art. Hybridoma cells may be produced by mixing the splenic lymphocytes with an appropriate fusion partner under conditions which will allow the formation of stable hybridomas. The antibody producing cells and fusion partner cells may be fused in polyethylene glycol at concentrations from about 30% to about 50%. Fused hybridoma cells may be selected by growth in hypoxanthine, thymidine and aminopterin supplemented Dulbecco's Modified Eagles Medium (DMEM) by procedures known in the art. Supernatant fluids may be collected from growth-positive wells and screened for antibody production by an immunoassay such as an Enzyme Linked Immunosorbent Assay. Hybridoma cells from antibody positive wells may be cloned by a technique such as the soft agar technique of MacPherson, Soft Agar Techniques or by limiting dilution in which hybridomas are diluted in suitable growth media and re-plated such that ˜1 hybridoma cell is pipetted into a single well of a 96-well plate where it grows over the course of 10 days as a single clone, in Tissue Culture Methods and Applications, Kruse and Paterson, Eds., Academic Press, 1973.

The term “identity”, as recognized by those skilled in the art, represents a comparison between two or more amino acid sequences performed using published methods and software known in the art. For example, the compared amino acid sequences are optimally aligned, and the number of amino acid differences are counted and converted to a percentage. For example, if a first amino acid sequence of 50 amino acids is optimally aligned with a second amino acid sequence of 50 amino acids, and 5 out of 50 amino acids differ from the second amino acid sequence, then the first amino acid sequence is said to have 10% identity with the second amino acid sequence. All sequences provided herein may have at least 90%, at least 91%, at least 92%, at least 93%, at least 94%, at least 95%, at least 96%, at least 97%, at least 98%, at least 99%, or 100% identity to the reference sequence.

A prioritized antibody refers to an antibody of particular interest for further investigation. An antibody may be prioritized based on computational methods (e.g., predicted 3D structures). Additionally or alternatively, an antibody may be prioritized based on preliminary experimental results. A prioritized antibody is considered to be comparatively more likely (e.g., more likely than other tested antibodies) to perform well neutralizing a protein.

A disease of interest may refer to an infection caused by a pathogen, such as a bacterium, virus, or other microorganism. A disease of interest may be a fast mutating or fast evolving disease. In exemplary embodiments, the disease is SARS-CoV-2.

In one aspect, an antibody is provided herein, the antibody comprising a variable heavy chain (VH) comprising SEQ ID NO: 1, 2, or 3 or a sequence having at least 90% identity thereto; and a variable light chain (VL) comprising SEQ ID NO: 4, 5, 6, 7, 8, 9, or 10 or a sequence having at least 90% identity thereto.

As used herein, the terms “protein” or “polypeptide” or “peptide” may be used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

In a second aspect, provided herein is a pharmaceutical composition comprising a therapeutically effective amount of at least one of the antibodies disclosed herein, and a pharmaceutically acceptable carrier.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the chemical compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The chemical compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. In some embodiments, the optimum effective amount can be readily determined by one skilled in the art using routine experimentation.

In a third aspect, provided herein is a method for treating coronavirus disease (COVID-19) in a subject in need thereof, the method comprising administering to the subject the pharmaceutical composition comprising at least one of the antibodies described herein. In exemplary embodiments, the COVID-19 is caused by a SARS-CoV-2 Delta variant.

As used herein, the term “administering”, refers to dispensing, delivering, or applying the therapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The terms “treating” and “to treat” includes the reducing, repressing, delaying or preventing COVID-19.

As used herein, the term “subject” may be used interchangeably with the term “patient” or “individual” and may include an “animal” and in particular a “mammal.” Mammalian subjects may include humans and non-human animals, such as other primates, domestic animals, farm animals, and companion animals such as dogs, cats, guinea pigs, rabbits, rats, mice, horses, cattle, cows, etc.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

One of the most demanding challenges in medical care and clinical investigation is to fight against constantly evolving pathogens and abnormal cells (such as cancer) under selective therapeutic pressure. Antibodies have accounted for most of the top therapeutic sales impacting medical treatments for immune diseases and cancer over the past years (1-3). However, the traditional development of any therapeutic drugs including antibodies is time and labor intensive, requiring a large scale of experimental screening and validation. Many antibody screening strategies for neutralization efficacy such as phage display, ribosome display, and mammalian cell surface display have relatively low efficiency. In this study, we set out to accelerate antibody development by experimental validations for broad-spectrum antiviral therapy, utilizing the fast-mutating severe acute respiratory syndrome coronavirus 2 (SARS-CoV-2) as a targeting model.

SARS-CoV-2 enters host cells via its viral spike protein binding with the host cell receptor angiotensin-converting enzyme 2 (ACE2) (5, 6), which is highly expressed on the cell membrane in various human organs including the lungs, heart, and kidney (7). During the pandemic of coronavirus disease 2019 (COVID-19), neutralizing antibodies became one of the earliest approaches that rapidly developed and effectively treated the early phase of infections before a vaccine was developed and before the coronavirus evolved to evade neutralization (8-12). Most of the coronavirus neutralizing antibodies disrupt the spike interactions with human ACE2 thereby preventing viral entry for prophylactic and therapeutic applications (8-15).

The constantly evolving viral sequences especially the variants of concern (Delta and Omicron) started to escape from neutralizing antibodies and vaccination immunity (16, 17), partially contributing to the death of nearly seven million people worldwide (18). For instance, the spike L452R and T478K mutations in the Delta variant, also known as B.1.617 lineage, are located at the periphery or the epitope region of the receptor binding domain (RBD) and are found to reduce antibody neutralizing activity (19, 20). The Omicron variant, which is characterized by the presence of around 32 mutations in the RBD, escapes most therapeutic neutralizing antibodies and largely vaccine-elicited antibodies (17).

To better prepare for any inevitable pandemics or epidemic diseases, rapid development and design of broad-spectrum antibodies would facilitate the early response against infectious pathogens (21). We used experimental data and validated patient antibodies against broad coronavirus variants.

### Results

Diversity of Sequenced Antiviral Antibodies from Human B Cells

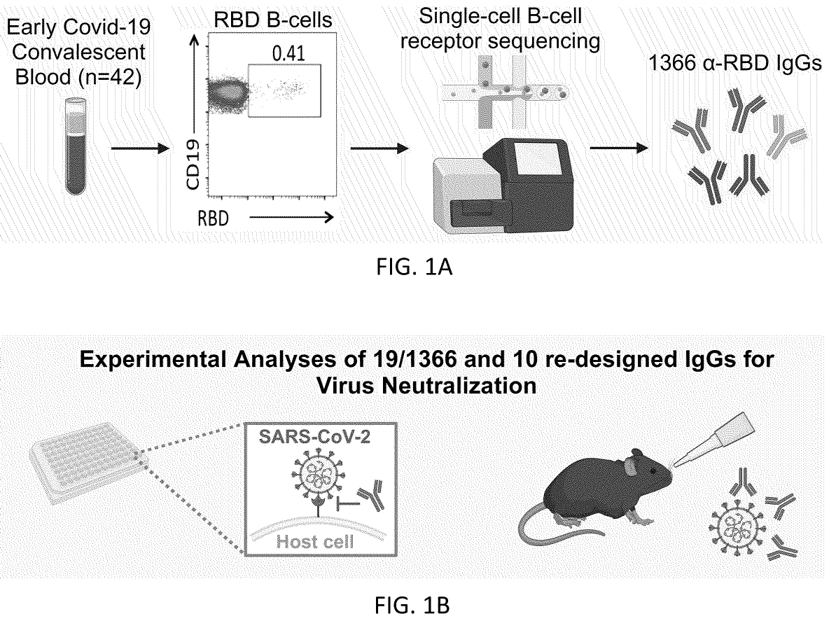

An overview of a study flow for investigating antibodies is shown in FIG. 1A. To identify and produce anti-spike antibodies, we first collect blood specimens from 42 patients recovered from early COVID-19 as described (24), flow sorted the RBD-bound IgM memory B cells for subsequent single-cell VDJ sequencing via the 5′ 10× genomics platform, and retrieved 1376 heavy-light chain pairs of IgG antibody sequences. We cloned 19 IgG pairs of heavy and light chains and a few computationally redesigned antibodies for experimental analyses and functional neutralization against SARS-CoV-2 infections in vitro and in vivo (FIG. 1B)

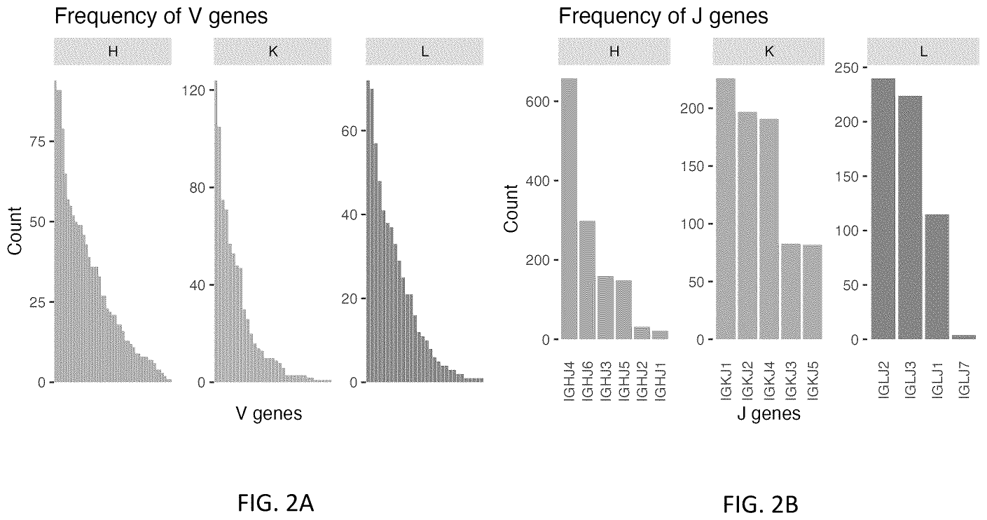

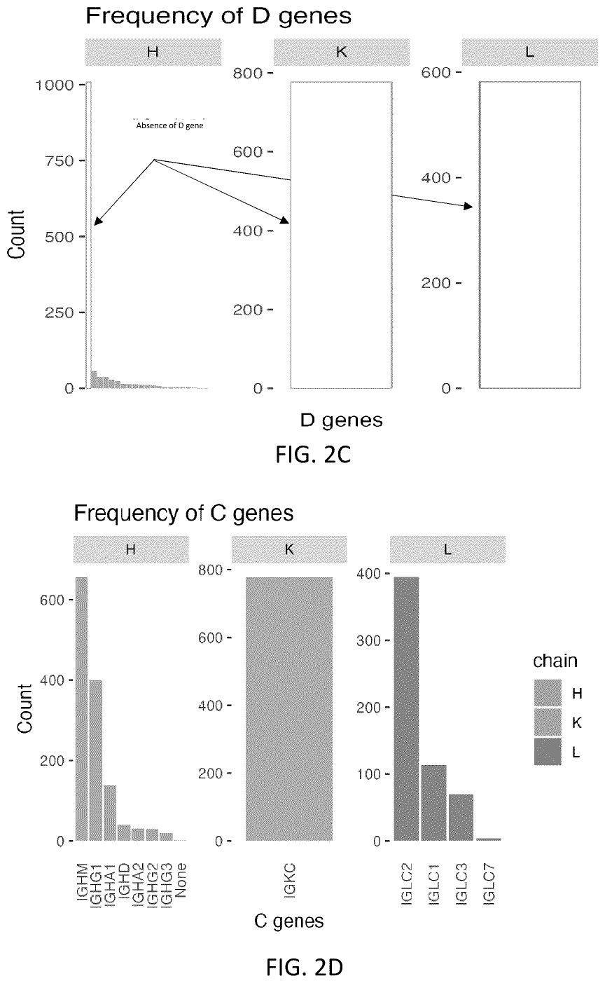

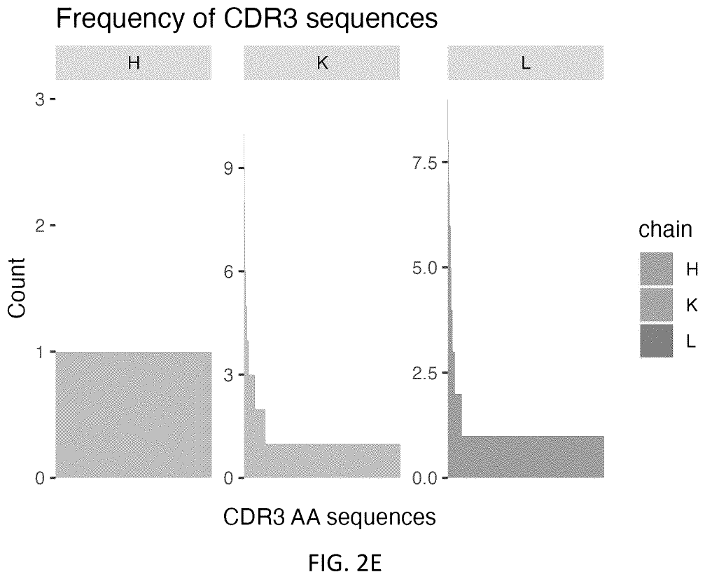

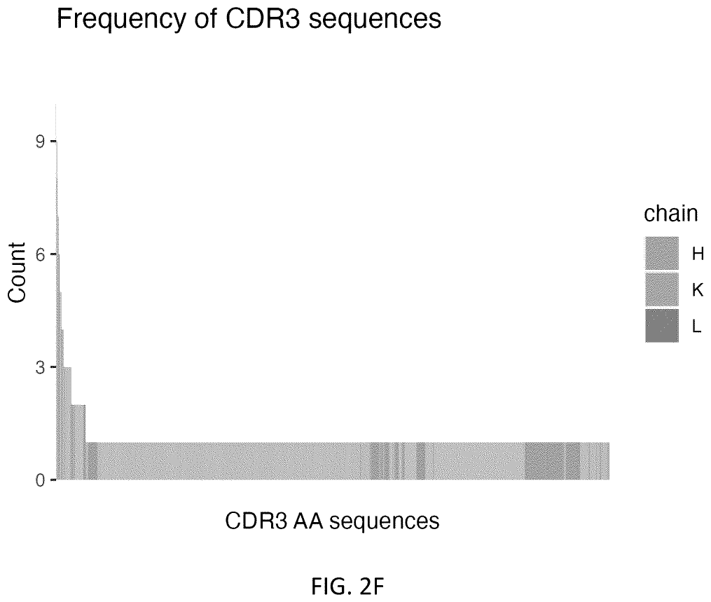

After obtaining 1376 heavy-light chain pairs of IgG antibodies using the CellRanger platform (10× genomics), we identified the individual V, D and J genes (sequences) for each antibody as well as the type of light chain (Kappa or Lambda). Frequency analyses of individual V and J genes revealed a non-even distribution, as certain V and J genes were more common with 60 to 600 counts than others with few counts (FIGS. 2A-2B). D genes were not detected in the light chains and were rarely seen in the heavy chain (FIG. 2C). The relative diversity of V genes was identified with the J genes skewed toward a few sequences, such as IGHJ4 of over 600 counts, suggesting that our sample group of antibody candidates share general properties of IgGs with distinct specificity of how each IgG recognizes and binds to spike-RBD. We detected a few C genes in the heavy chains and Lambda light chains but not within the Kappa chains (FIG. 2D). Every CDR3 sequence within the H chains was identified once, similar to most of the CDR3 sequences in K and L chains with only a small portion repeating 2-10 times (FIGS. 2E-2F).

### Functional Tests of IgG Candidates in Neutralizing RBD Binding and Viral Infections

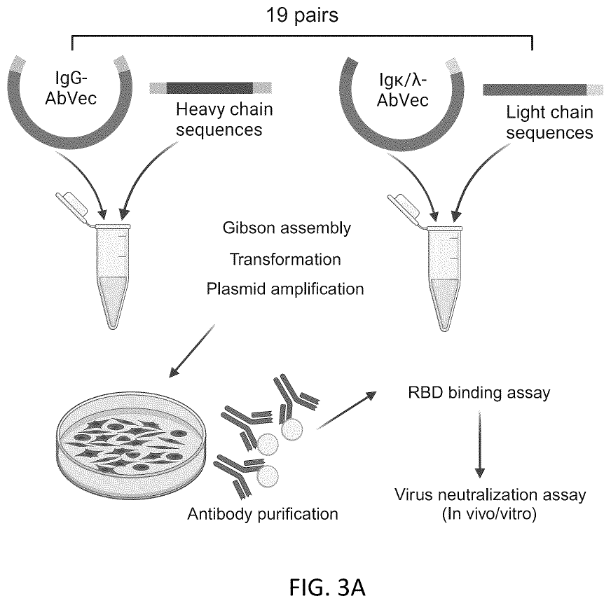

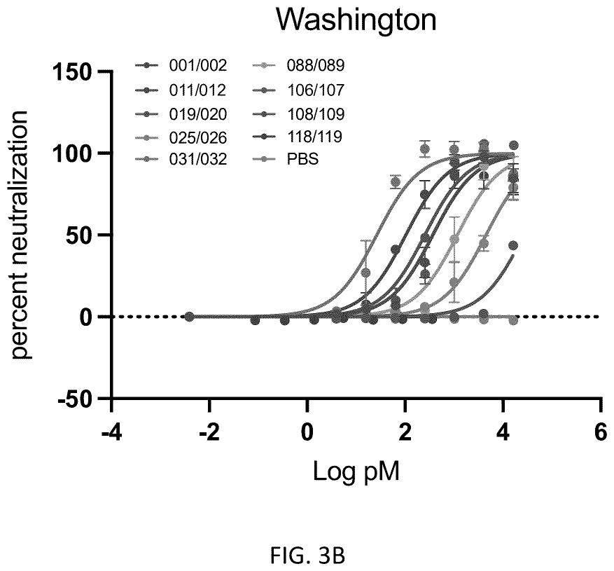

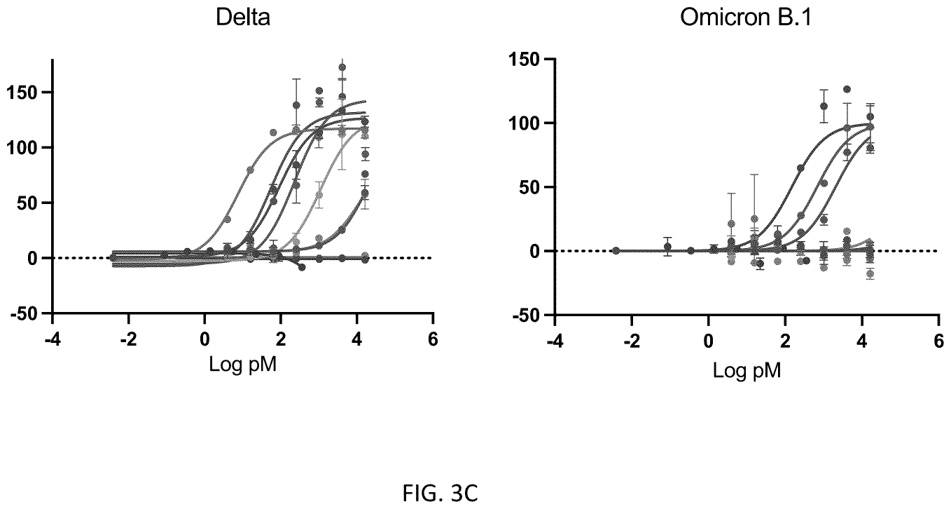

To assess IgG candidates in neutralizing WT and variant strains of SARS-CoV-2, we randomly selected 19 IgG antibodies for experimental tests (FIG. 3A). Following heavy and light chain cloning, the plasmid DNA was transfected into HEK293T cells for IgG overexpression, and the IgGs were purified for functional analyses in blocking RBD binding to host cells and neutralizing live viral infections.

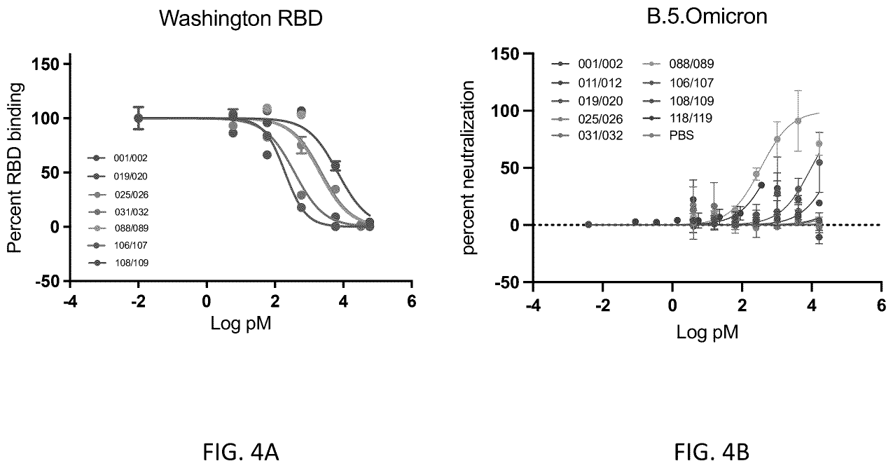

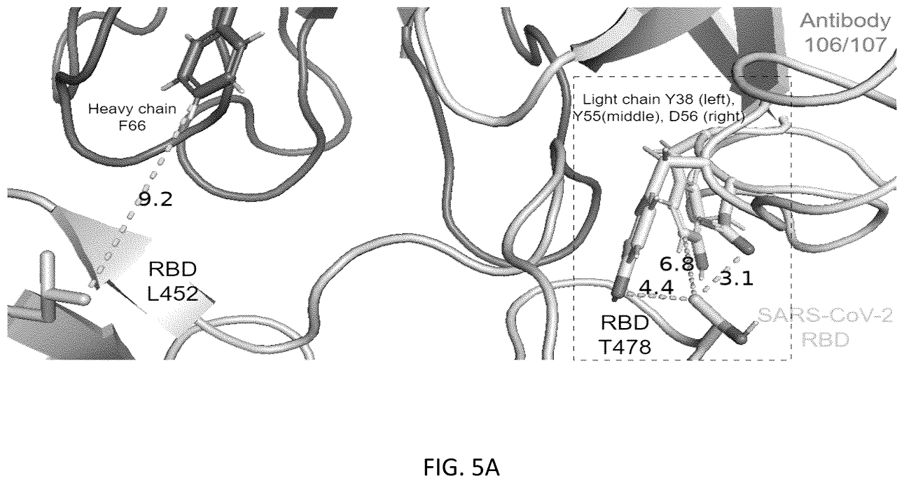

With flow cytometry-based measurement of RBD-binding to human ACE2 expressing cells (31), we evaluated the IgG abilities in blocking the RBD-binding to cellular ACE2 receptor (binding inhibition IC50) (FIG. 4A). Seven out of 19 randomly selected then tested IgGs (001/002, 019/020, 025/026, 031/032, 088/089, 106/107 and 108/109) inhibited the RBD binding to ACE2+ HEK293 cells in a dose dependent manner (FIG. 4A). Among the seven neutralizing IgGs, two were extremely potent (106/107 and 31/32) with IC50<0.5 nM. The rest 12 antibodies did not show any effective neutralization activity at the concentration tested.

Consistently, nearly half of the IgG antibodies had neutralizing effects at serially diluted concentrations (up to 16 nM) on live SARS-CoV-2 infection to ACE2 overexpressing A549 cells (FIG. 3B). Specifically, the IgG clones 106/107, 011/012 and 031/032 showed the lowest EC50 values against the WT Washington strain, demonstrating the strongest neutralizing effects (FIG. 3B). Interestingly, although the IgGs were derived from the COVID-19 patients infected with the WT Washington strain (before the variants of concern emerged and widespread), many of the IgGs possessed neutralizing capacities against the Delta and Omicron variants as well. In fact, three of the clones (001/002, 031/032, and 108/109) were about ten times more effective in neutralizing the Delta variant as compared to the wildtype (FIG. 3C, FIG. 4B).

Experimental data was used to validate the efficacy of the designed broad-spectrum antibodies. Two of the tested IgGs showed high potencies and efficacies in neutralizing RBD-binding and viral infection, representing a 67% success rate compared to 37% (7/19) from a random subset of antibodies.

### Redesign of an Antibody Improves Neutralizing the Delta Variant

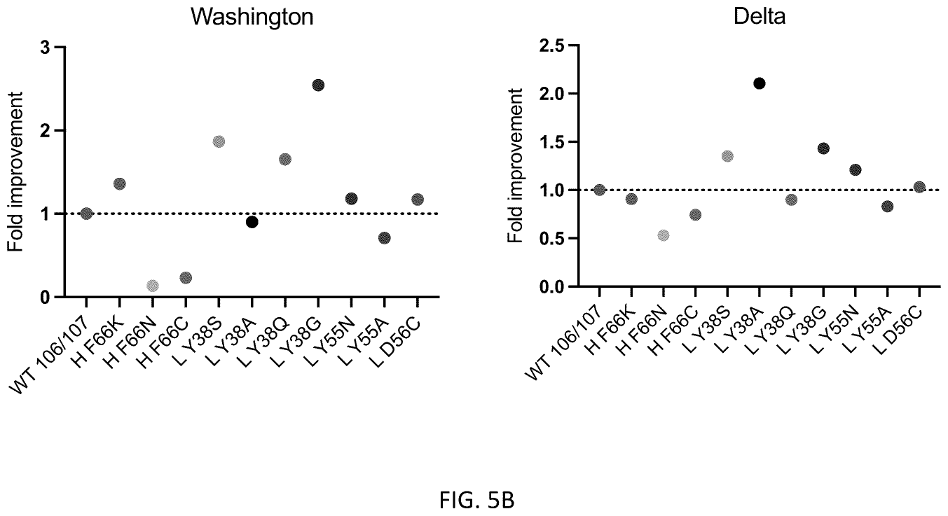

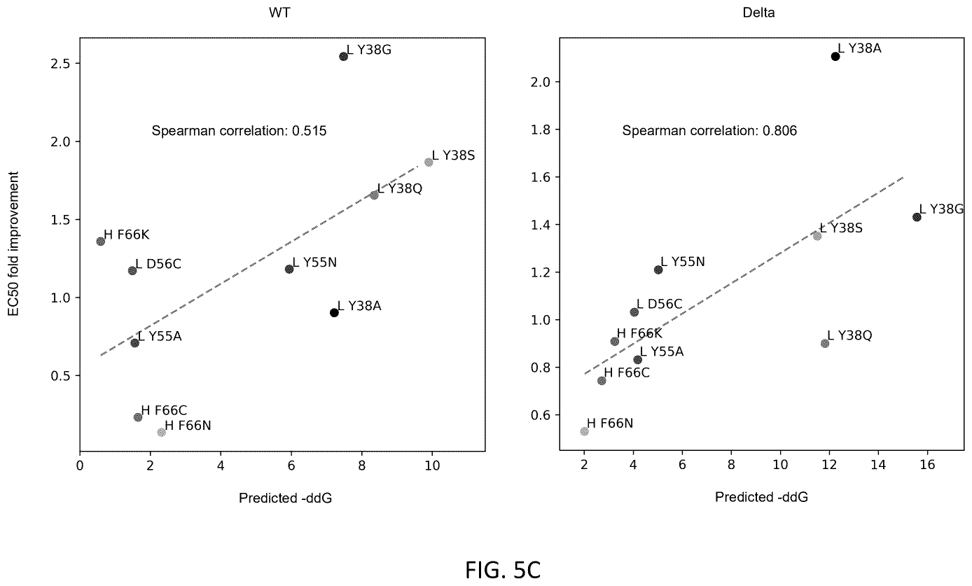

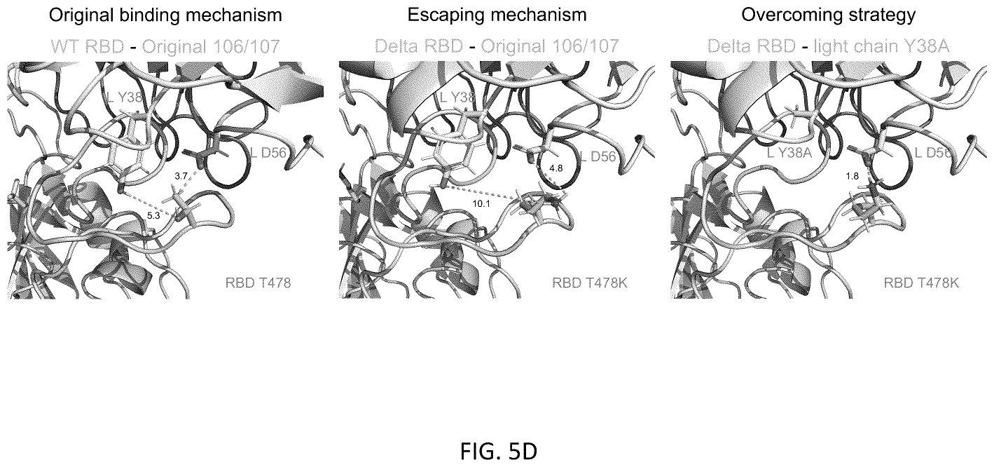

We picked IgG 106/107 and verified its neutralizing efficacy against WT and the Delta variant. Based on predicted IgG-RBD structures, we used a physics principle-driven multistate protein design program, iCFN (36) to computationally redesign IgG 106/107 to improve its binding to the Delta variant RBD without losing much affinity to the WT RBD. Ten single amino-acid substitutions near suggested binding sites of T478K were proposed, including three at the heavy chain (all on F66) and seven at the light chain (four on Y38, two on Y55 and one on D56) (FIG. 5A and Table 5). All four designs at Y38 stood out due to strongly improved electrostatic interactions, half being large-to-small apolar substitutions (Y38A and Y38G) and the other half hydrophobic-to-polar (Y38S and Y38Q).

TABLE 1
Computationally proposed top-designs (single amino-acid
substitutions) of antibody 106/107 and their predicted binding-energy
changes against the wild type or the Delta variant. The highlighted
designs Y38A (L) and Y38G (L), both hydrophobic substitutions,
were predicted to stabilize binding to the Delta variant the most. They
were both validated to improve neutralization of the Delta variant.

| | | Proposed | | |
|---|---|---|---|---|
| | | design | | Δ binding |
| Re- | Re- | (original | Δ binding | affinity |
| designed | designed | amino | affinity for | for delta |
| position | position | acid −> | WT RBD | RBD |
| [IMGT | [index in | proposed | (Kcal/mol; | (Kcal/mol; |
| index | PDB files | amino | excluding | excluding |
| (chain)] | (chain)] | acid ) | VdW) | VdW) |
| 66 (H) | 57 (H) | PHE −> LYS | −0.59 | −3.24 |
| 66 (H) | 57 (H) | PHE −> ASN | −2.32 | −2.01 |
| 66 (H) | 57 (H) | PHE −> CYS | −1.65 | −2.71 |
| 38 (L) | 149 (L) | TYR −> SER | −9.9 | −11.51 |
| 38 (L) | 149 (L) | TYR −> ALA | −7.22 | −12.26 |
| 38 (L) | 149 (L) | TYR −> GLN | −8.35 | −11.82 |
| 38 (L) | 149 (L) | TYR −> GLY | −7.48 | −15.57 |
| 55 (L) | 166 (L) | TYR −> ASN | −5.94 | −5.02 |
| 55 (L) | 166 (L) | TYR −> ALA | −1.56 | −4.18 |
| 56 (L) | 167 (L) | L 167 ASP −> CYS | −1.49 | −4.04 |

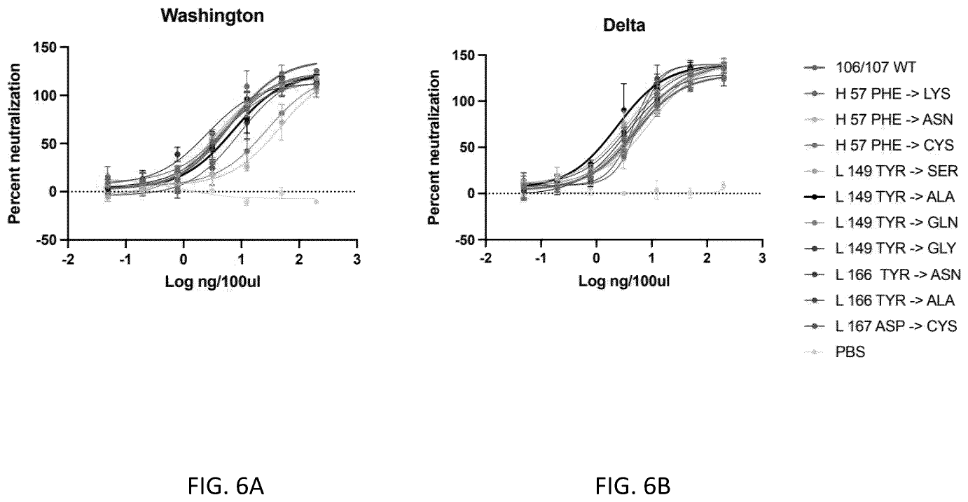

We cloned ten re-designed IgG 106/107m antibodies and measured their neutralization abilities against live viruses of the wild type and Delta variant. Strikingly, three of the four top-ranked designs, all at Y38 residue including Y38A, Y38S and Y38G (not Y38Q) showed most improved neutralization against the Delta variant, with Y38A of over 2-fold increase in neutralization efficacy (FIG. 5B and FIGS. 6A-6B). Importantly, their neutralization activities for the WT virus were not significantly impaired, which is consistent with the intended computational design. Our principle-driven protein design led to energy scores with positive to strongly positive ranking performances: the Spearman's rank correlation coefficient between the predicted binding energy changes and the measured pEC50 values for the 10 designs was 0.515 and 0.806 for wild type and the Delta variant, respectively (FIG. 5C). It also led to mechanistic explanations of Y38A's gained binding to the Delta RBD: T478K in the Delta variant may cause Y38 of the antibody 106/107 to be buried from the solvent and pay desolvation penalty to the binding affinity (FIG. 5D, middle panel), whereas the substitution to a small hydrophobic residue Y38A (as well as Y38G) in the antibody 106/107 can reduce such penalty (FIG. 5D, right panel). With the proof of concept from single substitution designs, we anticipate that higher-order redesigns or even de novo designs would further improve the broad-spectrum neutralization profile.

### Prioritized IgG Candidates Show Anti-Infection in Transgenic Mice

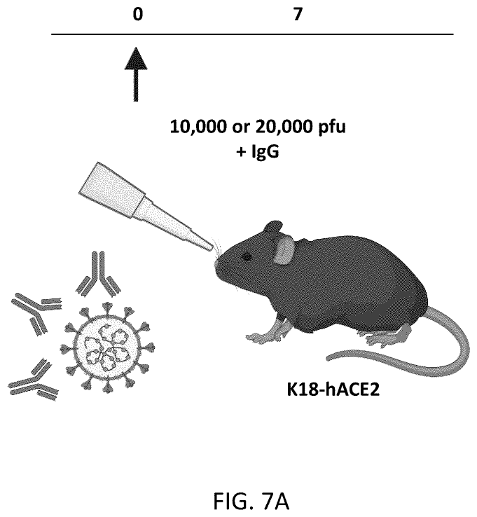

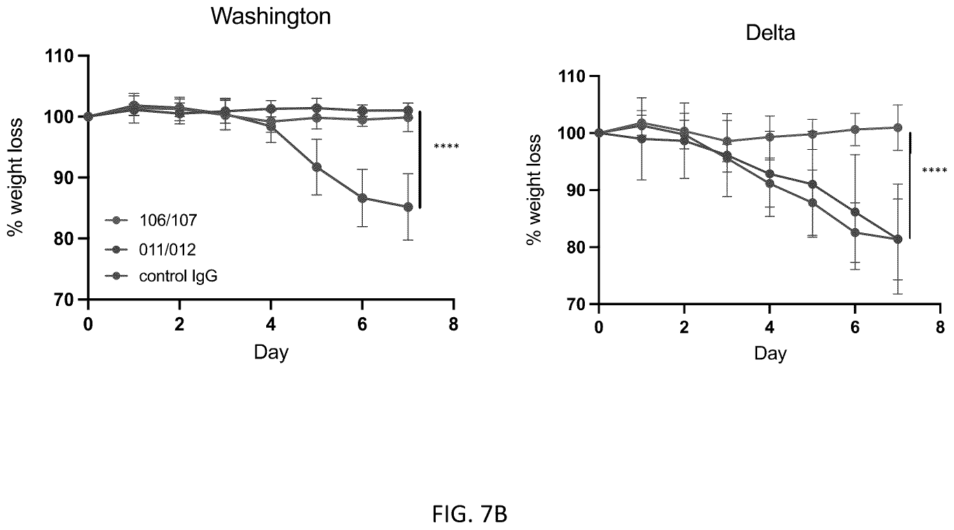

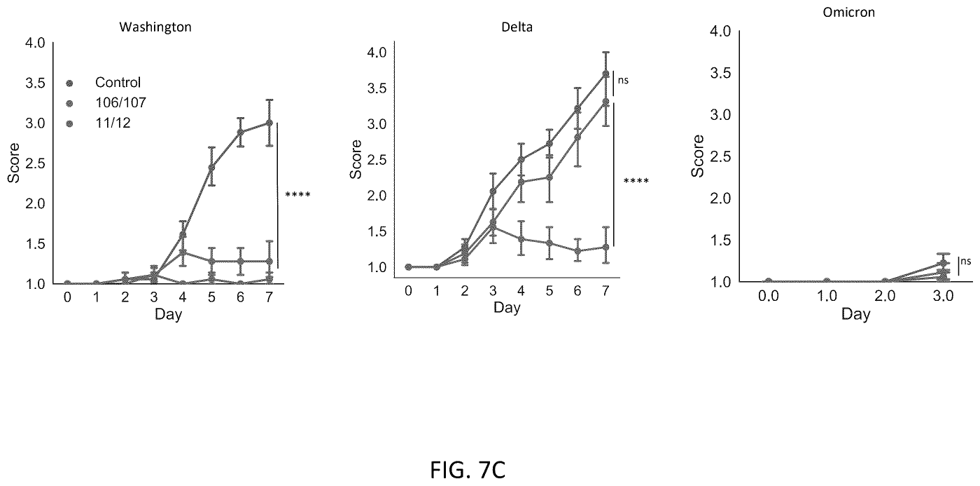

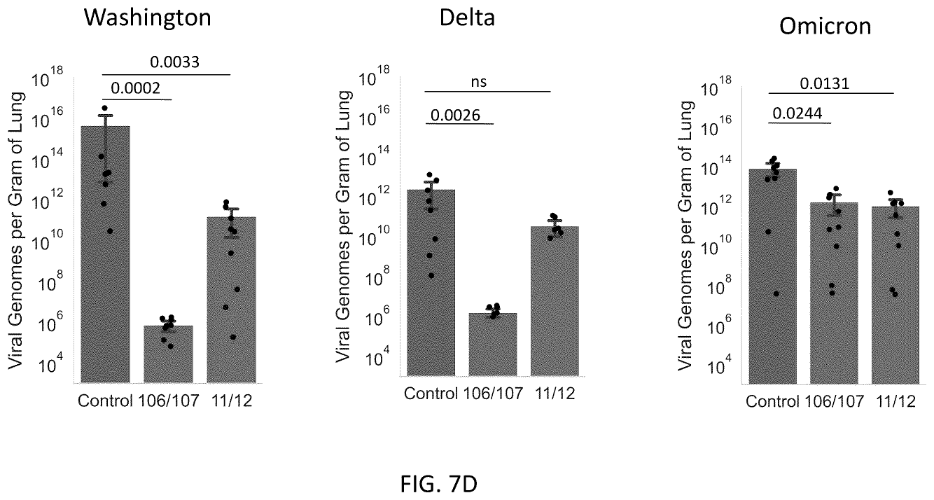

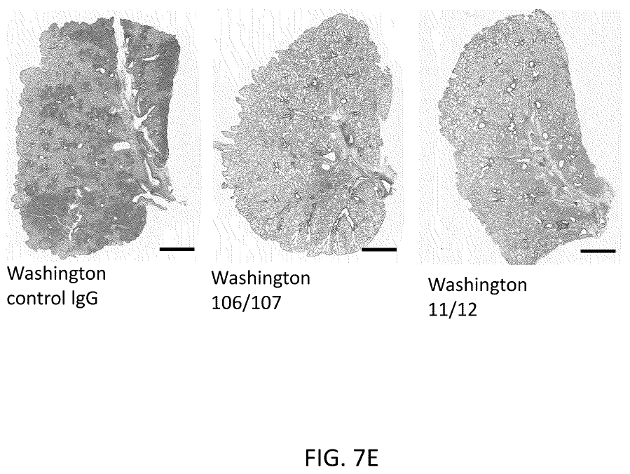

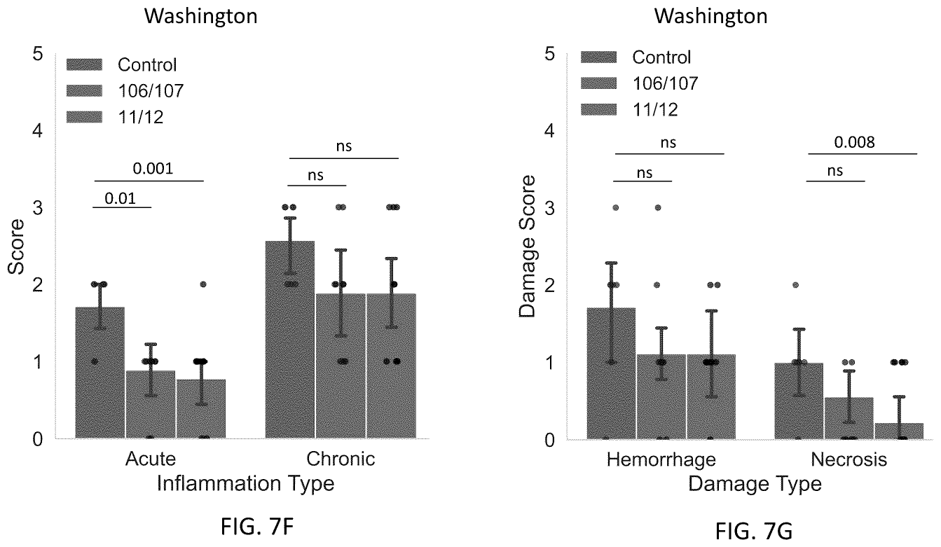

We next evaluated the therapeutic efficacy of two best neutralizing IgG candidates (106/107 and 011/012) prioritized by our experimental platform in their ability to prevent multi-strain SARS-CoV-2 infection in the well-established hACE2 transgenic mouse model in vivo (FIG. 7A). Antibodies were administered to mice by nasal inhalation to determine their effects on preventing viral infection of the three viral strains (WT Washington, Delta, and Omicron). In concordance with the in-vitro cell survival after viral infections, both antibodies prevented weight loss and health deterioration in mice infected with the Washington strain, whereas only IgG 106/107, but not IgG 011/012, prevented the mice from Delta variant infection-caused weight loss and clinical symptoms (FIGS. 7B-7C). Furthermore, the viral titers in the lungs of infected mice were significantly lower after the 106/107 antibody treatment which also neutralized the viral variants, consistent with the live virus neutralization results in vitro (FIG. 7D).

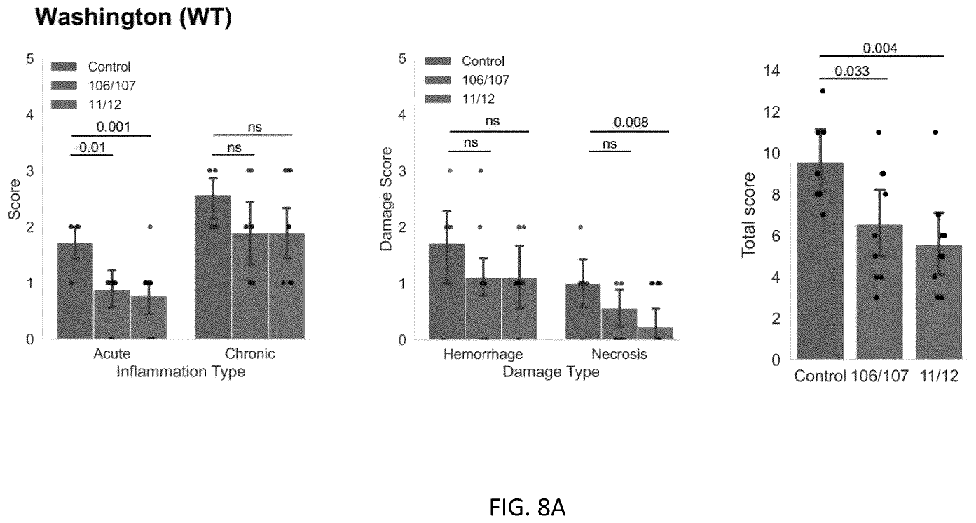

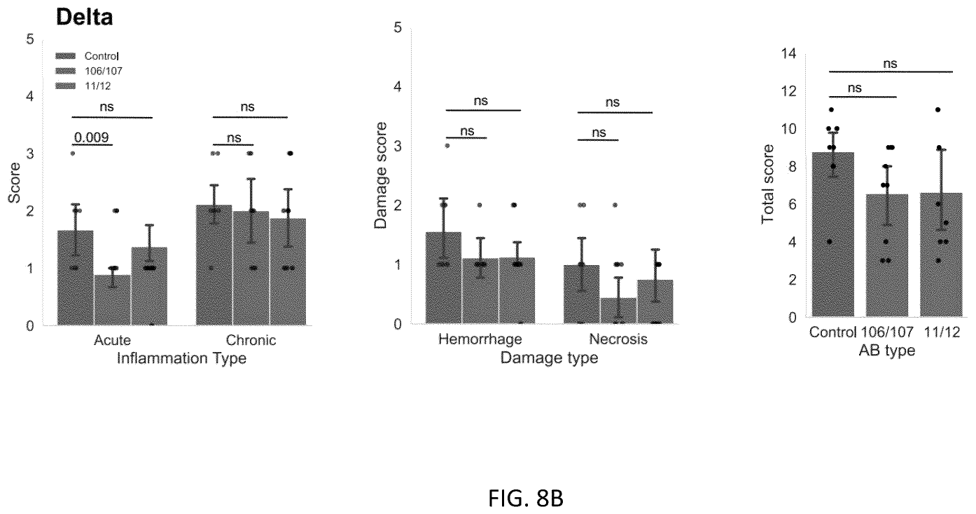

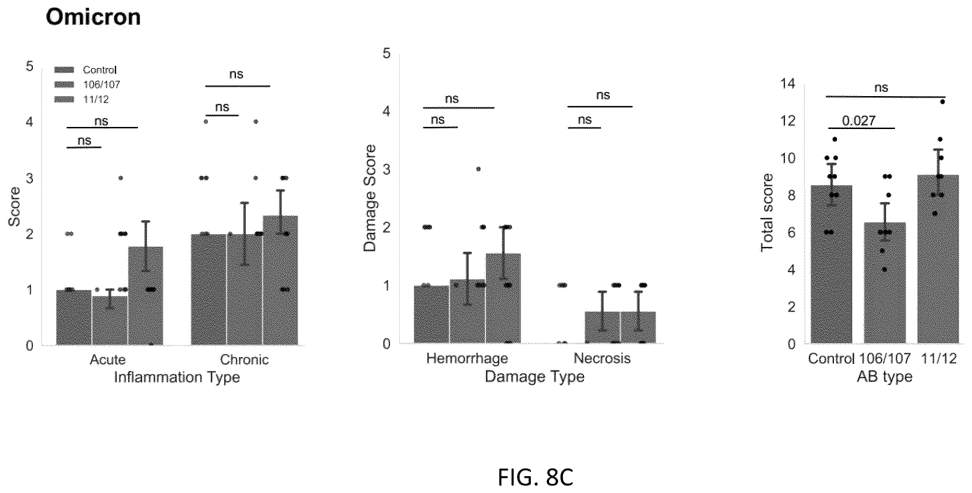

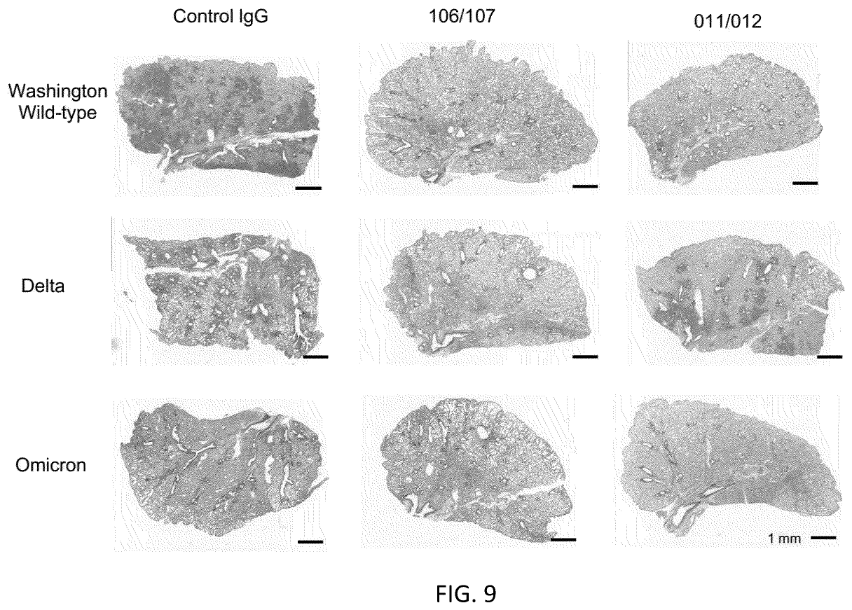

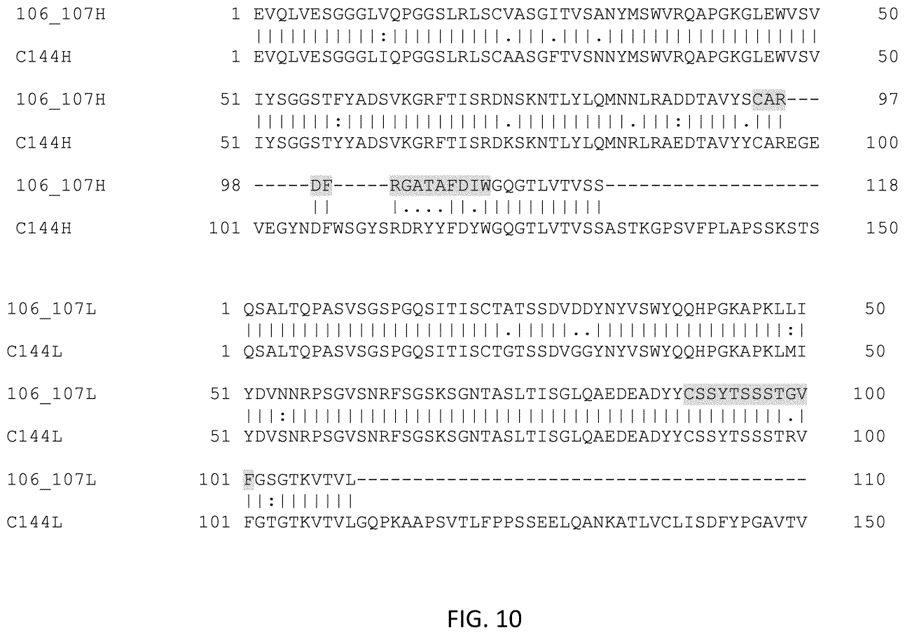

Notably, while the low pathogenic Omicron variant did not cause severe disease in mice, treatment with either IgG 106/107 or IgG 011/012 resulted in lower levels of viral titers in the lungs of the infected animals (versus the isotype IgG control group) (FIGS. 7C-7D). As expected, Omicron infection did not cause significant changes in clinical scores and lung histology even in the control mice without candidate treatment (FIGS. 7C-7D, FIGS. 8A-8C, and FIG. 9). Histology analyses of the lungs of infected mice reveal that the 106/107 and 011/012 neutralizing IgGs were able to reduce acute lung inflammation following infection with the Washington and Delta strains, albeit no significant effect on chronic inflammation (FIGS. 7E-7F, FIGS. 8A-8C, FIG. 9). Additionally, the IgG 011/012 antibody treatment significantly inhibited the necrosis in the lungs of mice infected with the Washington as compared to the control IgG (FIG. 7G). These data demonstrated the IgG prioritization strategies in pre-clinical studies.

### Discussion

Therapeutic antibody development faces the dual challenges of expensive experimental screening and constantly evolving targets under selective pressure. Using SARS-CoV-2 as a testing model, our extensive RBD-specific IgG sequencing on a sizable scale with COVID-19 patients in the early stages of the pandemic, reveals that a significant number of effective clones against the WT virus, lose effectiveness against the variants of concern, such as Delta and Omicron. We identified certain IgG antibody clones with potential enhancements in blocking variant infections in ACE2+ cells, confirmed through in vitro and in vivo virus infection studies. These findings offer a potential explanation for why certain COVID-19 patients exhibit greater resistance to reinfection by SARS-CoV-2 variants compared to others, as frequently observed (37).

We have demonstrated that replacement of a single residue spin in the IgG core for RBD binding with a smaller and hydrophobic alanine (Y38A) dramatically improves its neutralization activity against the Delta variant. This unexpected discovery also provides a rationale for us to harness the computational power in virtual IgG re-design for their use to treat/prevent emerging new pathogen variants or mutating oncogenic targets, which are urgent clinical demand. Over the last few years, while the convalescent plasma treatment has been proven to be effective in treating COVID-19 patients [30], almost all licensed monoclonal antibodies have eventually failed to neutralize the Omicron variant [31].

While many clinical antibodies are used to neutralize other pathogens, treat autoimmune diseases, and combat cancer, there is an urgent call to prepare efficacy-improving strategies in advance or in early response phase before therapy resistance develops.

### Methods and Materials

### Sex as a Biological Variable

Human blood specimens were collected from both male and female patients. Additionally, our study examined male and female mice, and similar findings are reported for both sexes.

### Human Subject Study and Biosafety Approvals for Blood Draws

Human blood specimens from convalescent COVID-19 patients and related research activities were implemented under NIH guidelines and the protocols approved by the Northwestern University Institutional Review Board (STU00205299) as well as the Institutional Biosafety Committee for COVID-19 research.

### B-Cell Sequencing (VDJ), Bioinformatic Analysis

B cells were isolated from blood of convalescent COVID-19 donors using the EasySep B kit (Stemcell Technologies, cat no 17954). SARS-nCoV-2 Spike RBD (Raybiotech, catalog no. 230-30162) was biotinylated via a commercially available kit (Thermo Scientific cat no. 21330) and the resulting biotin-labeled protein was purified through a Zeba quick spin column (Thermo Scientific cat no. 89882). The RBD-biotin was prebound with streptavidin-AlexaFluor-647 to make RBD-647 (Invitrogen, cat no. S21374). B cells were stained with CD19, CD27, CD38, anti-IgM and RBD-647 and a TotalSeq-C hashtag oligo (Biolegend). RBD-647+ B cells were sorted on a FACS Aria (BD Biosciences) in the Robert H. Lurie Cancer Center Flow Cytometry core facility. Due to the small number of RBD-647+B cells isolated from each patient, we combined B cells with monocytes isolated from the same patients, labeled with a different hashtag oligo (24).

B Cells and monocytes were partitioned using a 10× genomics Chromium Controller for GEM generation followed by single-cell library construction using 10× Chromium Next GEM Single Cell V (D) J Library reagent kit. Libraries were sequenced at the Northwestern University Sequencing Core Facility on an Illumina HiSeq 4000 using the sequencing parameters indicated by the manufacturer. We aligned the sequences aligned using CellRanger (10× genomics). Aligned antibody sequences from CellRanger were extracted for downstream analysis. VDJ sequences were ordered from IDT and cloned into the vector AbVec antibodies (Addgene).

### Antibody Re-Design Against the Delta Variant

We chose antibody 106/107 as the “seed” for improved neutralization against a SARS-CoV-2 variant (Delta). The highest-weight structural model of the antibody-RBD (WT) complex as the input, a computational protein design program iCFN (36) is used to first predict the antibody-RBD (Delta variant) complex structure and then design amino-acid substitutions for the antibody to gain neutralization against the Delta variant. Specifically, both steps involve multistate design with single substrate per state (see more details in (36)) and find the optimal structures (and sequences, when applicable) to minimize the energy difference between a positive and a negative state.

When predicting the antibody-RBD (Delta variant) complex structure, we maximally disrupt binding by minimizing the folding-energy difference between the separate (positive-state) and bound (negative-state) antibody-RBD, as detailed in (44). All residues within 5 Å of RBD residues L452 and T478 were treated flexible during design, while amino-acid substitutions L452K and T478R were enforced.

When designing the optimal single amino-acid substitutions for the antibody 106/107 and simultaneously predicting the structures, we maximally enhance RBD-binding by minimizing the folding-energy difference between the bound (positive-state) and the separate (negative-state) antibody-RBD while constraining the folding stability, as detailed in (36) (XRCC1 design). The antibody redesign positions are all residues within 5 Å of RBD residues 452 and 478, including 1 on the heavy chain near residue 452 of RBD and 7 on the light chain near residue 478 of RBD.

### Cloning of Antibody Expression Plasmids

The VDJ sequences of heavy and light chains of prioritized antibody pairs were obtained from the single cell VDJ sequencing. DNA fragments harboring the specific variable sequences were then synthesized by gBlock Gene Fragment technology by Integrated DNA Technologies (IDT). Synthesized double stranded DNA fragments were then cloned into AbVec2.0-IGHG1 (Addgene #80795), AbVc2.0-1.1-IGKC (Addgene #80796), and AbVec1.1-IGLC2-Xhol (Addgene #99575) to generate the heavy chain, kappa light chain, and lambda light chains, respectively. The IgG expression vectors and protocols were generously shared by Drs. Jenna Guthmiller and Patrick Wilson at the University of Chicago. Proper insertions of cloned DNA fragments were confirmed by Sanger sequencing.

### Antibody Production and Purification

Plasmids expressing the heavy and light chain of each tested antibody were transfected into HEK293T cells (ATCC CRL-3216) by a calcium chloride transfection method. Briefly, 60-80% confluent HEK293T cells were transfected with about 19 micrograms of each heavy and light chain expression plasmids (up to 1044 μL nuclease free H2O) mixed with 188 μL of 2M CaCl2. Followed by dropwise addition of 1.25 ml of 2×HBS buffer pH 7.12 (50 mM Hepes Acid, 280 mM NaCl, 1.5 mM NA2HPO4) while introducing bubbles to the mix. Following incubation for 20 minutes at room temperature, 15.5 mL cDMEM was added to the mix and subsequently transferred to cells and incubated at 37° C. 5% CO2 for 16 hours, after which cells were washed with 1×PBS and fresh cDMEM was added to cells and incubated for three more days. Supernatants were collected and spun at 2,000×g for 10 minutes at 4° C. to pellet cell debris. Antibodies were purified from culture supernatants by protein A agarose beads (Thermo Fisher Pierce™ Protein A Agarose, 20333). Briefly, cell culture supernatant was added to pre-washed beads (500 μL) and incubated on a table top rocker for 2 hours at room temperature followed by overnight incubation at 4° C. Bead-bound antibodies were collected by centrifugation at 1800×g for 10 minutes at 4° C. (Break off) and washed in 1M NaCl followed by two 1×PBS washes. Antibodies were eluted by adding 3 ml 0.1M glycine-HCL (pH 7.12) and rocking at room temperature for 10 minutes. Following centrifugation at 1800×g for 10 minutes at 4° C., supernatants containing the antibodies were neutralized by adding 200 ul 1M Tris-HCl (pH 8.8). Antibody solutions were then concentrated using an Amicon protein concentrator (4 mL capacity, 30 kDa MWT cutoff) and buffer exchanged with 1×PBS and antibodies were stored at 4° C.

### Quantification of Antibody Concentrations Using ELISA

The Human IgG ELISA Kit (ab 195215) was used to quantify the concentration of purified IgGs following the manufacturer's instructions and optical density was measured using the SpectraMax iD5 plate reader.

### Cell-Based Neutralization Assay

To create neutralized Spike Receptor Binding Domain (RBD), purified antibodies were incubated with the RBD-biotin-AF647 bait (3.3 nM) for 45 min on ice, then incubated with ACE2 expressing HEK-293 (ACE2+ HEK-293) cells (200,000 cells in 100 μL 2% extracellular vesicles (EV)-free FBS/PBS) for 45 min on ice. RBD bait that was incubated with PBS, or with non-fluorescent RBD bait (mock control) were used as controls. Cells were then washed twice with 2% EV-free/PBS (300×g for 5 min). Dapi was added to stain dead cells and analysis was performed using BD FACSymphony A5-laser analyser. Viable singlets were gated for percentage of the RBD-AF647+ population. Data were analyzed using Flow Jo v10.6.2. and GraphPad Prism 9.5.0.

### Live SARS-CoV-2 Virus Infection of A549 Cells (BSL3)

The live virus neutralization experiments were conducted at the NIAID-supported BSL-3 facility at the University of Chicago Howard T. Ricketts Regional Biocontainment Laboratory.

One day prior to viral infection, A549 cells overexpressing human ACE2 (A549-hACE2) (obtained from tenOever and colleagues) (45) were seeded onto 96-well plates at a density of 10,000 cells per well. Antibody dilutions were made in Infection Media with 2% FBS. Antibody dilutions were mixed with 500 pfu of a SARS-CoV-2 strain, WT Washington (nCoV/Washington/1/2020), variant Delta (NR-55672 SARS-Related Coronavirus 2, Isolate hCoV-19/USA/MD-HP05647/2021 (Lineage B.1.617.2), variant Omicron (NR-56481 SARS-Related Coronavirus 2 Isolate hCoV-19/USA/GA-EHC-2811C/2021 (Lineage B.1.1.529) or variant Omicron Isolate hCoV-19/USA/COR-22-063113/2022 (Lineage BA.5) and incubated at 37° C. in the dark for 1 hour. Culturing media was then replaced with infected antibody dilutions and allowed to incubate for 72-96 hours or until positive control wells show at least 50% CPE (cytopathic effect). At which point the infectious media was removed and cells were fixed with 100 μL of Formalin solution (10% Formalin Fisher 23305510). Cells were incubated at room temperature with Formalin for at least 15 minutes to ensure viral inactivation. Then formalin media was removed, and cells were stained in 0.25% Crystal Violet solution (0.25% w/v Crystal Violet Sigma C0775 in 20% EtOH) for 15-30 minutes, after which the Crystal violet is washed off under gently flowing tap water. Plates were then allowed to dry uncovered on the benchtop for at least 24 hours prior to analysis using the Infinite 200 Pro TECAN plate reader. Control wells such as no virus mock control in addition to a non-antibody treated control were included. A value of maximal death caused by the virus was evaluated from the virally infected but non-treated control wells while the maximal normal cell growth was determined from the mock-infected control wells. The average absorbance value of the non-treated control wells was subtracted from the remaining absorbance values to establish “0” values for non-treated wells. Next, all absorbance values were divided by the average absorbance value of the mock-infected control thereby setting the value of the mock-infected control to 100.

### Mouse Experiments

Three experimental groups of 15 week-old hACE2 transgenic B6.Cg-Tg (K18-ACE2) 2Prlmn/J (K18-hACE2) mice were set up with nine mice per group (4 or 5 females and males each group to reach about 50%/50%). Mice were housed in specific pathogen-free facilities at the BSL-2 facility at University of Chicago Howard T. Ricketts Regional Biocontainment Laboratory. Three groups of IgG (1.2 ug for Washington and Delta or 8.5 ug for Omicron) were each mixed with SARS-CoV-2 viral strains; Washington at 10,000 pfu, Delta at 10,000 pfu, or Omicron at 20,000 pfu and incubated for 1 hour at 37° C. After incubation, the mice were anesthetized with isoflurane and 25 μL of the mixture were administered intranasally. Following viral infection challenges, animals were monitored for health twice daily and weighed once per day. Clinical scoring system included: Score 0 (pre-inoculation)—mice are bright, alert, active, normal fur coat and posture. Score 1 (post-inoculation, pi)—mice are bright, alert, active, normal fur coat and posture, no weight loss. Score 1.5—mice present with slightly ruffled fur but are active OR weight loss might occur but does not reach 2.5%; recovery can be expected. Score 2 (pi)-ruffled fur OR less active OR<5% weight loss; recovery might occur. Score 2.5 (pi)-ruffled fur OR not active but movies when touched OR hunched posture OR difficulty breathing OR weight loss 5-10%; recovery is unlikely but still might occur. Score 3 (pi)-ruffled fur OR inactive but moves when touched OR difficulty breathing OR weight loss at 11-20%; recovery is not expected. Score 4 (pi)-ruffled fur OR positioned on its side or back OOR dehydrated OR difficulty breathing OR weight loss>20% OR labored breathing; recovery is not expected. Score 5 (pi)-death. Three days post infection, mice challenged with the Omicron variant were euthanized while the mice challenged with Washington and Delta variant were euthanized on day seven post infection. One lung was used to determine SARS CoV-2 viral genome levels and the other lung was fixed with formalin.

### Lung Fixation and Histopathological Analysis

Lung tissue was submerged in 1 mL formalin for 48 hours. Formalin was removed and 1 mL formalin was added and incubated for an additional 12 days. Tissue was tested for viral inactivation and released. Fixed lungs were then embedded in paraffin and sectioned by routine procedures followed by H&E staining. Stained slides were scanned then analyzed using the NDP view2 software. Double-blinded evaluation of the percent of total lung surface area involvement was performed by a pathologist following a graded scheme adopted from a previous report (46).

Lung Viral Genome Level Measurement Using qRT-PCR

RNA was extracted from mouse lungs using the Nucleospin 96 RNA extraction kit as per written instructions (Macherey-Nagel 740709.4). Prior to being run through the binding columns, the lung tissue was collected in the R1A buffer provided with the kit and homogenized using a FastPrep fp120 homogenizer with 1.4 mm ceramic beads (Omni international SKU 19-645). RNA was eluted in RNAse free water and used for qRT-PCR. Which was performed using an Applied BioSystems Step One Plus Realtime PCR system, using the SupperScriptIII Platinum One-Step qRT-PCR Kit with ROX (Invitrogen 11745-500). Sample RNA was measured using a standard curve made by extracting viral RNA from lab viral stocks. The quantity of RNA in the viral stock was measured with a Nanodrop 2000. CDC recommended N2 primers and probe used in the qRT-PCR were purchased from IDT (10006824, 10006825, and 10006826).

TABLE 2
Informal Sequence Listing

| SEQ ID NO and | |
|---|---|
| Description | Sequence |
| SEQ ID NO: 1 | MGWSCIILFLVATATGVHSEVQLVESGGGLVQPGGSLRLSCVASGI |
| (Heavy Chain | VSANYMSWVRQAPGKGLEWVSVIYSGGSTKYADSVKGRFTISRDNT |
| 106 - F77K) | SKNTLYLQMNNLRADDTAVYSCARDFRGATAFDIWGQGTMVTVSSA |
| (corresponds to | STKGPSVFPLAPSSKSTSGGTAALGCLVKDYFPEPVTVSWNSGALT |
| IMGT Index 66) | SGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKS |
| | LSLSPGK* |
| SEQ ID NO: 2 | MGWSCIILFLVATATGVHSEVQLVESGGGLVQPGGSLRLSCVASGI |
| (Heavy Chain | TVSANYMSWVRQAPGKGLEWVSVIYSGGSTNYADSVKGRFTISRDN |
| 106 - F77N) | SKNTLYLQMNNLRADDTAVYSCARDFRGATAFDIWGQGTMVTVSSA |
| (corresponds to | STKGPSVFPLAPSSKSTSGGTAALGCLVKDYFPEPVTVSWNSGALT |
| IMGT Index 66) | SGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKS |
| | LSLSPGK* |
| SEQ ID NO: 3 | MGWSCIILFLVATATGVHSEVQLVESGGGLVQPGGSLRLSCVASGI |
| (Heavy Chain | TVSANYMSWVRQAPGKGLEWVSVIYSGGSTCYADSVKGRFTISRDN |
| 106 - F77C) | SKNTLYLQMNNLRADDTAVYSCARDFRGATAFDIWGQGTMVTVSSA |
| (corresponds to | STKGPSVFPLAPSSKSTSGGTAALGCLVKDYFPEPVTVSWNSGALT |
| IMGT Index 66) | SGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKS |
| | LSLSPGK* |
| SEQ ID NO: 4 | MGWSCIILFLVATATGVHSQSALTQPASVSGSPGQSITISCTATSS |
| (Light Chain | DVDDYNSVSWYQQHPGKAPKLLIYDVNNRPSGVSNRFSGSKSGNTA |
| 107 - Y53S) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 38) | TTPSKQSNNKYAASSYLSLTPEQWKSHRSYSCQVTHEGSTVEKTVA |
| | PTECS* |
| SEQ ID NO: 5 | MGWSCIILFLVATATGVHSQSALTQPASVSGSPGQSITISCTATSS |
| (Light Chain | DVDDYNAVSWYQQHPGKAPKLLIYDVNNRPSGVSNRFSGSKSGNTA |
| 107 - Y53A) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 38) | TTPSKQSNNKYAASSYLSLTPEQWKSHRSYSCQVTHEGSTVEKTVA |
| | PTECS* |
| SEQ ID NO: 6 | MGWSCIILFLVATATGVHSQSALTQPASVSGSPGQSITISCTATSS |
| (Light Chain | DVDDYNQVSWYQQHPGKAPKLLIYDVNNRPSGVSNRFSGSKSGNTA |
| 107 - Y53Q) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 38) | TTPSKQSNNKYAASSYLSLTPEQWKSHRSYSCQVTHEGSTVEKTVA |
| | PTECS* |
| SEQ ID NO: 7 | MGWSCIILFLVATATGVHSQSALTQPASVSGSPGQSITISCTATSS |
| (Light Chain | DVDDYNGVSWYQQHPGKAPKLLIYDVNNRPSGVSNRFSGSKSGNTA |
| 107 -Y53G) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 38) | TTPSKQSNNKYAASSYLSLTPEQWKSHRSYSCQVTHEGSTVEKTVA |
| | PTECS* |
| SEQ ID NO: 8 | MGWSCIILFLVATATGVHSQSALTQPASVSGSPGQSITISCTATSS |
| (Light Chain | DVDDYNYVSWYQQHPGKAPKLLINDVNNRPSGVSNRFSGSKSGNTA |
| 107 - Y70N) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 55) | TTPSKQSNNKYAASSYLSLTPEQWKSHRSYSCQVTHEGSTVEKTVA |
| | PTECS* |
| SEQ ID NO: 9 | MGWSCIILFLVATATGVHSQSALTQPASVSGSPGQSITISCTATSS |
| (Light Chain | DVDDYNYVSWYQQHPGKAPKLLIADVNNRPSGVSNRFSGSKSGNTA |
| 107 - Y70A) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 55) | TTPSKQSNNKYAASSYLSLTPEQWKSHRSYSCQVTHEGSTVEKTVA |
| | PTECS* |
| SEQ ID NO: 10 | MGWSCIILFLVATATGVHSQSALTQPASVSGSPGQSITISCTATSS |
| (Light Chain | DVDDYNYVSWYQQHPGKAPKLLIYCVNNRPSGVSNRFSGSKSGNTA |
| 107 - D71C) | SLTISGLQAEDEADYYCSSYTSSSTGVFGSGTKVTVLGQPKANPTV |
| (corresponds to | TLFPPSSEELQANKATLVCLISDFYPGAVTVAWKADSSPVKAGVET |
| IMGT Index 56) | TTPSKQSNNKYAASSYLSLTPEQWKSHRSYSCQVTHEGSTVEKTVA |
| | PTECS* |
| SEQ ID NO: 11 | MGWSCIILFLVATATGVHSEVQLVESGGGLVQPGGSLRLSCVASGI |
| (Heavy Chain | TVSANYMSWVRQAPGKGLEWVSVIYSGGSTFYADSVKGRFTISRDN |
| L106) | SKNTLYLQMNNLRADDTAVYSCARDFRGATAFDIWGQGTMVTVSSA |
| | STKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKS |
| | LSLSPGK* |
| SEQ ID NO: 12 | MGWSCIILFLVATATGVHSQSALTQPASVSGSPGQSITISCTATSS |
| (Light Chain | DVDDYNYVSWYQQHPGKAPKLLIYDVNNRPSGVSNRFSGSKSGNTA |
| L107) | SLTISGLQAEDEADYYCSSYTSSSTGVFGSGTKVTVLGQPKANPTV |
| | TLFPPSSEELQANKATLVCLISDFYPGAVTVAWKADSSPVKAGVET |
| | TTPSKQSNNKYAASSYLSLTPEQWKSHRSYSCQVTHEGSTVEKTVA |
| | PTECS* |
| SEQ ID NO: 13 | CARDFRGATAFDIW |
| (Heavy Chain | |
| L106 CDR3) | |
| SEQ ID NO: 14 | CSSYTSSSTGV |
| (Light Chain | |
| L107 CDR3) | |

1. An antibody comprising:
a variable heavy chain (VH) comprising SEQ ID NO: 11 or a sequence having at least 90% identity thereto; and
a variable light chain (VL) comprising SEQ ID NO: 12 or a sequence having at least 90% identity thereto;
wherein the antibody comprises at least one mutation at a position selected from: position 77 relative to SEQ ID NO: 11; position 53 relative to SEQ ID NO: 12; position 70 relative to SEQ ID NO: 12; and position 71 relative to SEQ ID NO: 12.

2. The antibody of claim 1, wherein the VH comprises a lysine, an asparagine, or a cysteine at position 77 relative to SEQ ID NO: 11.

3. The antibody of claim 1, wherein the VL comprises a serine, an alanine, a glutamine, or a glycine at position 53, relative to SEQ ID NO: 12.

4. The antibody of claim 1, wherein the VL comprises an asparagine, or an alanine at position 70 relative to SEQ ID NO: 12.

5. The antibody of claim 1, wherein the VL comprises a cysteine at position 71 relative to SEQ ID NO: 12.

6. The antibody of claim 1,
wherein the VH comprises SEQ ID NO: 1, 2, 3, 11 or a sequence having at least 90% identity thereto; and
wherein the VL comprises SEQ ID NO: 4, 5, 6, 7, 8, 9, 10, 12 or a sequence having at least 90% identity thereto.

7. A pharmaceutical composition comprising a therapeutically effective amount of at least one antibody of claim 1; and a pharmaceutically acceptable carrier.

8. A method for treating coronavirus disease in a subject in need thereof, the method comprising administering to the subject the pharmaceutical composition of claim 7.

9. The method of claim 8, wherein the coronavirus disease is caused by a SARS-CoV-2 Delta variant.


SEQUENCE LISTING (14 sequences)

SEQ ID NO: 1 - protein, 467 aa, synthetic construct
MGWSCIILFLVATATGVHSEVQLVESGGGLVQPGGSLRLSCVASGITVSANYMSWVRQAPGKGLEWVSVIYSGGSTKYADSVKGRFTISRDNSKNTLYLQMNNLRADDTAVYSCARDFRGATAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 467 aa, synthetic construct
MGWSCIILFLVATATGVHSEVQLVESGGGLVQPGGSLRLSCVASGITVSANYMSWVRQAPGKGLEWVSVIYSGGSTNYADSVKGRFTISRDNSKNTLYLQMNNLRADDTAVYSCARDFRGATAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 467 aa, synthetic construct
MGWSCIILFLVATATGVHSEVQLVESGGGLVQPGGSLRLSCVASGITVSANYMSWVRQAPGKGLEWVSVIYSGGSTCYADSVKGRFTISRDNSKNTLYLQMNNLRADDTAVYSCARDFRGATAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 235 aa, synthetic construct
MGWSCIILFLVATATGVHSQSALTQPASVSGSPGQSITISCTATSSDVDDYNSVSWYQQHPGKAPKLLIYDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 235 aa, synthetic construct
MGWSCIILFLVATATGVHSQSALTQPASVSGSPGQSITISCTATSSDVDDYNAVSWYQQHPGKAPKLLIYDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 235 aa, synthetic construct
MGWSCIILFLVATATGVHSQSALTQPASVSGSPGQSITISCTATSSDVDDYNQVSWYQQHPGKAPKLLIYDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 235 aa, synthetic construct
MGWSCIILFLVATATGVHSQSALTQPASVSGSPGQSITISCTATSSDVDDYNGVSWYQQHPGKAPKLLIYDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 235 aa, synthetic construct
MGWSCIILFLVATATGVHSQSALTQPASVSGSPGQSITISCTATSSDVDDYNYVSWYQQHPGKAPKLLINDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 235 aa, synthetic construct
MGWSCIILFLVATATGVHSQSALTQPASVSGSPGQSITISCTATSSDVDDYNYVSWYQQHPGKAPKLLIADVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 235 aa, synthetic construct
MGWSCIILFLVATATGVHSQSALTQPASVSGSPGQSITISCTATSSDVDDYNYVSWYQQHPGKAPKLLIYCVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 467 aa, synthetic construct
MGWSCIILFLVATATGVHSEVQLVESGGGLVQPGGSLRLSCVASGITVSANYMSWVRQAPGKGLEWVSVIYSGGSTFYADSVKGRFTISRDNSKNTLYLQMNNLRADDTAVYSCARDFRGATAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 235 aa, synthetic construct
MGWSCIILFLVATATGVHSQSALTQPASVSGSPGQSITISCTATSSDVDDYNYVSWYQQHPGKAPKLLIYDVNNRPSGVSNRFSGSKSGNTASLTISGLQAEDEADYYCSSYTSSSTGVFGSGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 14 aa, synthetic construct
CARDFRGATAFDIW

SEQ ID NO: 14 - protein, 11 aa, synthetic construct
CSSYTSSSTGV